(19) United States
(12) Patent Application Publication
(10) Pub. No.: US 2018/0371392 A1
(43) Pub. Date: Dec. 27, 2018

(54) INLAY FOR A CULTURE PLATE AND CORRESPONDING METHOD FOR PREPARING A CULTURE PLATE SYSTEM WITH SUCH INLAY

(71) Applicant: CureVac AG, Tübingen (DE)

(72) Inventors: Christian MAYER, Pfullingen (DE); Isabel REICHERT, Schlaitdorf (DE)

(21) Appl. No.: 16/063,621

(22) PCT Filed: Dec. 21, 2015
(86) PCT No.: PCT/EP2015/080884

§ 371 Date: Jun. 18, 2018

(51) Int. Cl. C12M 1/00; C12M 3/00

(52) U.S. Cl. CPC C12M 23/34; C12M 1/18; C12M 3/00

(57) ABSTRACT

The present invention is concerned with an inlay (10) for a culture plate (20) for microorganisms or cells, a culture plate unit (30) for microorganisms or cells comprising such inlay (10), a culture system for microorganisms or cells comprising such culture plate unit (30), and a method for preparing a culture plate system (40) for microorganisms or cells with such inlay (10). Further, several uses as described herein are part of the present invention. The grid structure (11) is configured to fit into the culture plate (20) and is provided with a plurality of openings (12). The openings (12) are angled. In an embodiment, the openings (12) of the inlay (10) may be rectangular or square.

### FIELD OF THE INVENTION

The present invention relates to an inlay for a culture plate for microorganisms or cells, a culture plate unit for microorganisms or cells comprising such inlay, a culture system for microorganisms or cells comprising such culture plate unit, and a method for preparing a culture plate system for microorganisms or cells with such inlay. Further, several uses as described herein are part of the present invention.

### BACKGROUND OF THE INVENTION

In microbiology, transformation comprises the transfer of (foreign) nucleic acids (e.g., RNA, DNA, plasmids) into competent microorganisms, such as bacteria. Common transformation techniques comprise chemical transformation heat-shock transformation and electro-shock transformation. After transformation, bacteria that received foreign nucleic acids are commonly referred to as transformants. Similarly, in cell biology, transfection comprises the transfer of nucleic acids (e.g., RNA, DNA, plasmids) into eukaryotic cells, such as human cells, insect cells or mouse cells. Common transfection techniques comprise chemical transfection, electroporation, particle-based transfection or viral based transfection. After transfection, eukaryotic cells that received foreign nucleic acids are commonly referred to as transfectants.

For downstream analyses, it is essential to identify microorganisms or cells that received the transferred nucleic acid and originate from a single transformant or transfectant and are thus genetically identical. A common approach is to grow an appropriate dilution of a transfected culture on a selective solid medium. Subsequently, discrete colonies, consisting of genetically identical microorganisms or cells, serve as source for the inoculation of larger cultures (cf. colony picking).

High throughput (HT) cloning, which often involves parallel cloning of various different plasmid constructs, is increasingly used to generate cDNA libraries, BAC libraries, genomic DNA libraries, mutant libraries, or construct libraries. In HT cloning approaches, several transformation reactions are ideally handled in parallel. Therefore, a culture plate system containing an appropriate solid culture medium should be designed in a way that the risk of cross-contamination during the transfer of cells onto the plates is minimized. This is commonly ensured by partitioning of a culture plate into an array of discrete wells. Such culture plates are available in different sizes, wherein the shape of the wells is usually circular.

However, such conventional culture plate units may be improved.

### SUMMARY OF THE INVENTION

The present invention solves the above need, inter alia by providing an improved inlay for a culture plate for microorganisms or cells, a culture plate unit comprising such inlay, a culture plate system comprising such culture plate unit, a method for preparing such culture plate system and several uses of above mentioned devices, wherein the inlay is in particular improved to reduce a consumption of culture plates. In a first aspect, the present invention is directed to an inlay for a culture plate for microorganisms or cells comprising a grid structure. The grid structure is configured to fit into the culture plate and is provided with a plurality of openings. The openings are angled.

According to the invention, an exploitation of a culture plate surface is improved as the shape of the wells or openings in the culture plate are changed from circular to angled. The openings of the inlay may be rectangular or square. As a consequence, a larger portion of the total surface area of the culture plate is useable, which leads to a reduced consumption of culture plates and therefore less consumable waste. Thereby, e.g. a plating and screening of transformants/transfectants used in HT-cloning procedures becomes more economic based on less material consumption and reduced labour costs, as well as more ecologic based on less consumable waste. Further, a likelihood to obtain a sufficient number of discrete colonies in one well is increased and, as a consequence, costs are further reduced. Furthermore, an effective area used for plating and picking of colonies is strongly increased compared to conventional circular wells.

For example, the area that can be utilized for e.g. HT-cloning is increased by the angled openings according to the invention by 69.1% compared to a conventional 24 well culture plate with circular openings.

In an embodiment, the openings of the inlay may be rectangular or square. However, the openings may also be polygonal. In an embodiment, the openings of the inlay are through holes. However, the openings may also be blind holes.

In an embodiment, all openings of the plurality of openings of the inlay are similar in size. In the same or another embodiment, all openings of the plurality of openings are similar in shape. By the wordings “similar in size” and/or “similar in shape” essentially the same size and/or shape of the entrance of the opening in the inlay is meant. “Similar in size” and/or “similar in shape” comprise embodiments in which one or a few of the openings differ from the bulk of openings by e.g. a geometrical adaption to an irregular form of the culture plate. However, the openings of one inlay may also be identical or differ in view of their size and/or shape.

In an embodiment, the openings are regularly distributed relative to the culture plate. The wording “regularly distributed” means that the openings form a regular pattern in and on the culture plate. This can be achieved by e.g. providing the openings with similar distances between centre points of adjacent openings. However, the openings of one inlay may also differ be irregularly distributed relative to the culture plate.

In an embodiment, the grid structure is configured to partition the culture plate into an array of discrete wells. In an embodiment, the grid structure comprises longitudinal struts and transversal struts to partition the culture plate into an array of discrete wells. In this case, the wording “the grid structure is configured to fit into the culture plate” means that face sides of the longitudinal struts and transversal struts are configured to contact sidewalls of the culture plate when the grid structure is fitted into the culture plate. In another embodiment, the grid structure comprises longitudinal struts and transversal struts and additionally a frame unit to partition the culture plate into an array of discrete wells. The frame unit may surround the grid formed by the struts and transversal struts. In this case, the wording “the grid structure is configured to fit into the culture plate” means that lateral walls of the frame unit are configured to contact sidewalls of the culture plate when the grid structure is fitted into the culture plate. The longitudinal struts and the transversal struts may be arranged parallel to the frame unit's lateral walls or with an angle to the frame unit's lateral walls. For example, the longitudinal and the transversal struts may be arranged diagonal relative to the frame unit's lateral walls.

In an embodiment, the grid structure is unitarily made as one piece. This means, the grid structure may be taken as such or as one piece to be inserted in or removed from the culture plate. However, the longitudinal struts, the transversal struts and/or the frame unit may also be single pieces or compounds of pieces, which may be individually inserted in or removed from the culture plate. The first option may be easier to handle and may allow reducing costs and waste. The second option may be easier adapted to different culture plates or modes of operation.

In an embodiment, the grid structure has a height similar to a height of the culture plate. In other words, the height of the grid structure is such that it equals the height of the culture plate when it sits in the culture plate. This means, the longitudinal struts, the transversal struts and/or the frame unit of the grid structure may have at least partially the same height as the sidewalls of the culture plate. This may provide the advantage that for certain applications (e.g. HT-plating of microorganisms using a liquid handling device), a certain height of the culture plate is not exceeded. The longitudinal struts, the transversal struts and/or the frame unit of the grid structure may have at least partially a height in the range of 5 mm to 15 mm and in particular of 10 mm. However, other heights of the grid structure or the inlay may be beneficial for other types of application.

In an embodiment, the grid structure has a wall thickness in a submillimeter range. This means the longitudinal struts, the transversal struts and potentially also the frame unit may at least partially have a wall thickness between e.g. 0.1 mm and 0.9 mm. The wall thickness depends on the material. For example, for polyamide, a wall thickness of the grid structure may amount to 0.7 mm, and for acrylic, a wall thickness of the grid structure may amount to 0.5 mm. However, the wall thickness of the grid structure may also be in a range of 0.1 mm and 1.5 mm or in a range of 0.1 mm and 1.9 mm.

In an embodiment, the culture plate is dimensioned according to dimensions defined by the Society for Biomolecular Screening (SBS) according to ANSI standard.for culture plates and the grid structure is configured to fit into such culture plate. Thereby, the culture plate fits to most commercially available devices for e.g. plating of microorganisms/cells and/or picking of colonies, as most commercially available devices are designed for these dimensions. However, the inlay may be also designed to fit into culture plates with other dimensions.

In an embodiment, the culture plate comprises six openings, wherein each or most of the openings have a length of about 37.3 mm and a width of about 35.5 mm. In an embodiment, the culture plate comprises twelve openings, wherein each or most of the openings have a length of about 23.3 mm and a width of about 18.2 mm. In an embodiment, the culture plate comprises 24 openings, wherein each or most of the openings have a length of about 18.5 mm and a width of about 17.6 mm. In an embodiment, the culture plate comprises 48 openings, wherein each or most of the openings have a length of about 13.4 mm and a width of about 11.2 mm. In an embodiment, the culture plate comprises 96 openings, wherein each or most of the openings have a length of about 8.6 mm and a width of about 8.1 mm. For these openings, the wall thickness of the grid structure may be about 0.7 mm.

In an embodiment, the grid structure comprises a guiding element configured to contact the culture plate. This guiding element can be configured to contact the culture plate to guide the grid structure into the culture plate to ease an insertion of the grid structure into the culture plate. Therefore, one of the guiding element and the culture plate may form a groove and tongue system or the like for each other.

In an embodiment, the grid structure comprises a latching element configured to contact the culture plate. This latching element can be configured to contact the culture plate to better attach or grip the grid structure to the culture plate. Therefore, the latching element may be hook-shaped, barb-shaped, pointed or the like.

In an embodiment, the grid structure comprises a spike element configured to contact the culture plate. This spike element can be configured to contact the culture plate to reduce the contact with the culture plate to ease a removal of the grid structure from the culture plate. Therefore, the spike element may be a series of tips or the like.

In an embodiment, the grid structure comprises an orientation element configured to contact the culture plate. This orientation element of the inlay can be configured to allow an unequivocal orientation of the inlay in the culture plate. The orientation element of the inlay can also be configured to match to a culture plate orientation element of the culture plate, which may be used to allow an unequivocal orientation of the culture plate in e.g. an autoclave. In both cases, the orientation element of the grid structure may be a chamfer, a recess, a protrusion or the like.

In an embodiment, the grid structure is made of plastic as e.g. PMMA, PP, PET, PVC, polyamide, polyester, polystyrol, acrylic, polycarbonate or the like. For example, it may be made of PA2200 or UV Curable Acrylic Plastic. It can also be made of biodegradable plastics, starch-based plastics, cellulose-based plastics, polylactic acid (PLA), Poly-3-hydroxybutyrate (PHB), Polyhydroxyalkanoates (PHA). The grid structure may also be made of a compound material, ceramic, glass, gelatin, metal or an alloy. For example, it may be made of platinum, palladium, aluminium, magnesium or steel. In an embodiment, the grid structure is autoclavable. In an embodiment, the inlay is reusable, which allows reducing costs and waste.

In a second aspect, the present invention is directed to a culture plate unit for microorganisms or cells comprising a culture plate and an inlay as described above. In other words, the present invention is directed to a combination of inlay and culture plate. In an embodiment, the inlay is removeable from the culture plate, in particular after use of the culture plate. In this case, the inlay may be re-used in the same or another culture plate, particularly after the inlay and/or the plate has been autoclaved. However, the inlay and the culture plate can also be unitarily made from one piece.

The culture plate may be curved, which means either the entire surface of the culture plate may be curved or at least a sub-surface within an opening of the culture plate may be curved. The inlay and in particular its struts and optionally the frame may be adapted to fit to both options of a curved culture plate. The inlay may also be provided as a box comprising struts arranged on a bottom to be inserted in the culture plate. Then, the bottom or the inlay may be adapted to fit to both options of a curved culture plate.

In a third aspect, the present invention is directed to a culture plate system for microorganisms or cells comprising a culture plate, an inlay and a medium for growing a culture of microorganisms or cells. In an embodiment, the medium is thermosetting. The medium may be liquid or semi-solid or viscous when being filled into the culture plate and harden to a solid state by time (see the definition below for “solid medium”), temperature and/or pressure.

It is noted that the medium is of course adapted to the method, in which the culture plate system is used. This e.g. relates to medium suitable for the growth of the specific microorganisms or cells used e.g. in high throughput cloning or suitable for the growth of the microorganisms or cells to be detected. This furthermore relates to an optional selection that is employed in particular during high throughput cloning.

In a specific embodiment, said solid medium is agar growth medium. For microorganisms such as e.g. E. coli, such solid medium may be LB agar as commonly known to the skilled person. For cells and in particular eukaryotic cells, such as e.g. stem cells, soft agar may be used. Such a soft agar may e.g. be 1% agarose in medium suitable for growth of the respective eukaryotic cells.

In a specific embodiment, said solid medium is selective, in particular for specific microorganisms or cells, such as e.g. for microorganisms or cells comprising exogenous DNA with a selection marker. Thus, if said selection marker is a protein that confers resistance to an antibiotic, said antibiotic is added to the solid medium.

Thus, in this specific embodiment, an antibiotic such as e.g. ampicillin and/or kanamycin is comprised in concentrations routinely used for this purpose in the solid medium (e.g. 100 μg/ml ampicillin, or 50 μg/ml kanamycin). Alternatively, if e.g. colour distinction is (additionally) used and the selection marker is β-galactosidase or a subunit or derivative thereof, the solid medium will typically comprise in the routinely used concentrations i) a compound suitable for the induction of the β-galactosidase-gene, preferably Isopropyl-β-thiogalactopyranosid (IPTG) (e.g. 0.1 mM IPTG), and ii) a dye-substrate for the β-galactosidase or a subunit or derivative thereof, preferably X-Gal (e.g. 20 μg/ml X-Gal), that will be processed into the blue dye 5,5′-Dibromo-4,4′-Dichloro-Indigo.

Further media that can be used are described below.

In a fourth aspect, the present invention is directed to a method for preparing a culture plate system for microorganisms or cells with an inlay. The method for preparing a culture plate system comprises the following steps, not necessarily in this order:
a) filling a culture plate with a fluid medium, and
b) inserting an inlay into the culture plate.

The inlay comprises a grid structure, which is configured to fit into the culture plate. The grid structure is provided with a plurality of openings and the openings are angled. In an embodiment, the openings of the inlay may be rectangular or square.

In an embodiment, the grid structure comprises longitudinal struts and transversal struts to partition the culture plate into an array of discrete wells. In another embodiment, the grid structure comprises longitudinal struts and transversal struts and additionally a frame unit to partition the culture plate into an array of discrete wells. In an embodiment, the grid structure is unitarily made as one piece.

The method for preparing a culture plate system may be executed in the order a) and b) or b) and a).

In an embodiment, in the first case, the inlay is inserted into the culture plate filled with medium at a temperature near solidification of the medium. The temperature near solidification of the medium may be between 30 and 55° C. or between 40 and 45° C. In an embodiment, when preparing a culture plate, the temperature and/or the medium are selected to be near solidification of the medium. Both embodiments for its own overcome a drawback of commercially available culture plates with circular pre-exisiting wells in the culture plates, which is the fact that bulges of medium (e.g., agar medium) are formed at the transition between the medium and the wall of the well or opening (concave meniscus). Such bulges are caused by shrinking of the hot medium filled into the wells and subsequent drying of the medium during the solidification process. The bulges may cause unfavourable artefacts in the images used by e.g. image assisted colony detection and/or picking devices often used in HT-cloning procedures. This drawback is overcome by placing the inlay into the culture plate at a lower temperature just before solidification leading to less subsequent shrinking once the medium sticks to the walls. As a result, optical artefacts generated by e.g. agar bulges are minimized which optimizes a visual detection of e.g. colonies using image assisted automated picking devices.

In a fifth aspect, the present invention is directed to the use of an inlay or a culture plate as described above in a method for preparing a culture plate system as described above.

In a sixth aspect, the present invention is directed to the use of a culture plate system as described above in a method for obtaining at least one discrete colony from microorganisms or cells comprised in a solution. In an embodiment, the at least one discrete colony is obtained from microorganisms during high throughput cloning. In an embodiment, the at least one discrete colony is obtained from cells during transfection of eukaryotic cells. In an embodiment, the method is an automated method.

In a seventh aspect, the present invention is directed to the use of a culture plate system as described above in a method for determining the presence and/or quantity of microorganisms or cells potentially comprised in a solution. In an embodiment, the method is an automated method.

### BRIEF DESCRIPTION OF THE DRAWINGS

The figures shown in the following are merely illustrative and shall describe the present invention in a further way. These figures shall not be construed to limit the present invention thereto.

FIG. 1 shows a schematic illustration of an inlay for a culture plate for microorganisms or cells according to the invention.

FIG. 2 shows a schematic 3D illustration of another inlay according to the invention.

FIG. 3 shows a schematic illustration of a culture plate unit for microorganisms or cells according to the invention.

FIG. 4 shows a schematic illustration of a method for preparing a culture plate system for microorganisms or cells according to the invention.

FIG. 5A shows a schematic illustration of medium filled into a preexisting opening (or well) of a culture plate according to the prior art.

FIG. 5B shows in contrast a schematic illustration of an opening (or well) resulting from the inventive use of the inlay according to the invention, wherein liquid medium is first filled into a culture plate, followed by the addition of the inventive inlay and solidification of the medium.

FIG. 6A shows an image of two columns of a commercially available 24 well plate comprising round wells with medium after plating of bacteria.

FIG. 6B shows an image of two columns of a rectangular 24 well plate with medium prepared using the inventive inlay after plating of bacteria.

### DEFINITIONS

For the sake of clarity and readability the following definitions are provided. Any technical feature mentioned for these definitions may be read on each and every embodiment of the invention. Additional definitions and explanations may be specifically provided in the context of these embodiments.

As used in the specification and the claims, the singular forms of “a” and “an” also include the corresponding plurals unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group which preferably consists of these embodiments only.

The term “microorganisms” as used herein refers to microorganisms capable of forming colonies on solid medium, in particular to bacteria, fungi (e.g. yeasts) and single-celled eukaryotes. Most preferred microorganisms are selected from the group consisting of bacteria (e.g. E. coli), fungi (e.g. S. cerevisiae) and protists (e.g. xenic strains of Acanthamoeba).

The term “cells” as used herein in particular refers to eukaryotic cells, preferably human cells, mouse cells, monkey cells or insect cells. Said eukaryotic cells may also be stem cells and in particular undifferentiated stem cells. Further, said eukaryotic cells may also be cancer cells.

### Definitions and Aspects Relating to the Medium Used Herein

The term “solid medium” as used herein means a medium for microorganisms or cells, which does not allow for passive transfer of said microorganisms or cells within said medium but where a specific microorganism or cell transferred onto said solid medium will adhere to the spot of placement (if there is such an active mechanism) or simply stay on this spot once transferred there, e.g. in the form of a small volume of a liquid sample put onto a specific spot of said solid medium. The term “solid medium” further means that a liquid sample of a specific volume will adhere to the medium by the surface tension. The term “solid medium” thus includes all solid states (such as e.g. states of different viscosities) that are generally suitable for not allowing passive transfer within said medium and for holding a liquid sample at a specific spot. A particularly solid medium according to the present invention is agar, but semi-solid media and agars, respectively, are also encompassed, particularly for eukaryotic cells such as e.g. stem cells or cancer cells.

The term “selective” as used herein means that, after incubation of the medium comprising the microorganisms or cells, specific microorganisms or cells (e.g. comprising exogenous nucleic acid) can be distinguished from other microorganisms or cells (e.g. not comprising exogenous nucleic acid) in a suitable way (e.g. by survival or colour). A selection can also be based thereon that a specific insert is present or not in the exogenous nucleic acid comprised in the microorganisms or cells. The selectivity will be in favor of microorganisms comprising exogenous nucleic acid (or an insert therein), particularly if the selection is the viability (i.e. only microorganisms comprising exogenous nucleic acid or exogenous nucleic acid with an insert will survive and grow on the medium, and not the other way round in the meaning that only microorganisms not comprising exogenous nucleic acid or comprising exogenous nucleic acid without an insert will survive and grow on the medium). Compounds used for the selection are typically added to the medium.

The medium used in the culture plate system depends on the method carried out using said system. For high throughput cloning, it is in particular preferred to use microorganisms which are routinely used in laboratories for carrying out standard procedures. Corresponding media for these microorganisms are thus used.

For the aspect of the determination of the presence and/or quantity of microorganisms or cells capable of forming colonies on a solid medium, any microorganism or cell capable of forming colonies on solid medium can be detected as long as the growth conditions are known. These growth conditions comprise the solid medium suitable for growth of the microorganisms or cells.

The media suitable for growth of the following microorganisms or cells is known to the skilled person from text books and such media may be used in the context of the culture plate system of the present invention. Suitable media for the following microorganisms or cells may be used and are known to the skilled person:

Media for Protists as Microorganisms:

A protist is generally selected from the group consisting of the Amoebozoa (e.g. Tubulinae, Flabellinea, Stereomyxida, Acanthamoebidae, Entamoebida, Mastigamoebidae or Eumycetozoa), Archaeplastida (e.g. Glaucophyta, Rhodophyceae or Chloroplastida), Chromalveolata (e.g. Cryptophyceae, Haptophyta or Stramenopiles), Exavata (e.g. Fornicata, Parabasalia, Preaxostyla, Jakobida, Heterolobosea or Euglenozoa), Rhizaria (e.g. Cercozoa, Haplospodidia, Foraminifera or Radiolaria), and Opisthokonta (e.g. Mesomycetoza, Choanomonada or Metazoa). It can be preferred that said protists are selected from the group consisting of Chlorella; Chlamydomonas; Dunaliella; Haematococcus; Chorogonium; Scenedesmus; Euglena; xenic strains of Acanthamoeba, Naegleria, Hartmannella and Willaertia; and xenic strains of Vannella, Flabellula, Korotnevella, Paramoeba, Neoparamoeba, Platyamoeba and Vexillifera.

Media for Microorganisms:

Preferred bacteria are Escherichia coli, Corynebacterium (e.g. Corynebacterium glutamicum), Pseudomonas fluorescens, and Streptomyces (e.g. Streptomyces lividans). Most preferred can be Escherichia coli strains XL1-Blue, XL10-Gold, DH10B, DH5α, SURE, Stbl1-4, TOP10 and Mach1. Alternatively, said microorganisms are fungi, particularly yeasts, wherein Arxula adeninivorans (Blastobotrys adeninivorans), Yarrowia lipolytica, Candida boidinii, Saccharomyces cerevisiae, Schizosaccharomyces pombe, Hansenula polymorpha (Pichia angusta), Pichia pastoris, Aspergillus (e.g. Aspergillus oryzae), Trichoderma (e.g. Trichoderma reesei), and Myceliophthora thermophile are particularly preferred. Depending on the purpose, it can be most preferred to use Saccharomyces cerevisiae strains optimized for the analysis of interactions, such as e.g. yeast-two-hybrid-analyses.

The solid media may also be media suitable for the growth of pathogenic microorganisms. Exemplary pathogenic microorganisms are listed in the following, and the skilled person is aware of corresponding media for growth to be used in the culture plate system according to the present invention.

Media for Pathogenic Bacteria as Microorganisms:

Bacillus (e.g. Bacillus anthracis, Bacillus cereus); Bartonella (e.g. Bartonella henselae, Bartonella quintana); Bordetella (e.g. Bordetella pertussis); Borrelia (e.g. Borrelia burgdoferri, Borrelia garinii, Borrelia afzelii, Borrelia recurrentis); Brucella (e.g. Brucella abortus, Brucella canis, Brucella melitensis, Brucella suis); Campylobacter (e.g. Campylobacter jejuni); Chlamydia and Chlamydophila (e.g. Chlamydia pneumoniae, Chlamydia trachomatis, Chlamydophila psittaci); Clostridium (e.g. Chlostridium botulinum, Clostridium difficile, Clostridium perfringens, Clostdridium tetani); Corynebacterium (e.g. Corynebacterium diphtheriae); Enterococcus (e.g. Enterococcus faecalis, Enterococcus faecium); Escherichia (e.g. Escherichia coli); Francisella (e.g. Francisella tularensis); Haemophilus (e.g. Haemophilus influenzae); Heliobacter (e.g. Heliobacter pylori); Legionella (e.g. Legionella pneumophila); Leptospira (e.g. Leptospira interrogans, Leptospira santarosai, Leptospira weilii, Leptospira noguchii); Listeria (e.g. Listeria monocytogenes); Mycobacterium (e.g. Mycobacterium leprae, Mycobacterium tuberculosis, Mycobacterium ulcerans); Mycoplasma (e.g. Mycoplasma pneumoniae); Neisseria (e.g. Neisseria gonorrhoeae, Neisseria meningitidis); Pseudomonas (Pseudomonas areuginosa); Rickettsia (Rickettsia rickettsii); Salmonella (Salmonella typhi, Salmonella typhimurium); Shigella (e.g. Shigella sonnei); Staphylococcus (e.g. Staphylococcus aureus, Staphylococcus epidermidis, Staphylococcus saprophyticus); Streptococcus (e.g. Streptococcus agalactiae, Streptococcus pneumoniae, Streptococcus pyogenes); Treponema (e.g. Treponema pallidum); Ureaplasma (e.g. Ureaplasma urealyticum); Vibrio (e.g. Vibrio cholerae); Yersinia (e.g. Yersinia pestis, Yersinia enterocolitica, Yersinia pseudotuberculosis).

Media for Pathogenic Fungi as Microorganisms:

Candida (e.g. Candida species); Aspergillus (e.g. Aspergillus fumigatus, Aspergillus flavus, Aspergillus clavatus); Cryptococcus (e.g. Cryptococcus neoformans, Cryptococcus laurentii, Cryptococcus albidus, Cryptococcus gattii); Histoplasma (e.g. Histoplasma capsulatum); Stachybotrys (e.g. Stachybotrys chartarum).

The detection of a mold on corresponding medium is also possible. A mold is a fungus that grows in the form of multicellular filaments called hyphae. In contrast, fungi that can adopt a single celled growth habit are called yeasts. Molds are a large and taxonomically diverse number of fungal species where the growth of hyphae results in discoloration and a fuzzy appearance, especially on food. The network of these tubular branching hyphae, called a mycelium, is considered a single organism. Further relevant fungi are: Acremonium, Dematiaceae, Phoma, Alternaria, Eurotium, Rhizopus, Aspergillus, Fusarium, Scopulariopsis, Aureobasidium, Monilia, Stachybotrys, Botrytis, Mucor, Stemphylium, Chaetomium, Mycelia sterilia, Trichoderma, Cladosporium, Neurospora, Ulocladium, Paecilomyces, Wallemia, and Curvularia Penicillium.

Yeasts are eukaryotic microorganisms classified as members of the fungus kingdom with 1,500 species currently identified and are estimated to constitute 1% of all described fungal species. Yeasts are unicellular, although some species may also develop multicellular characteristics by forming strings of connected budding cells known as pseudohyphae or false hyphae. Most yeasts reproduce asexually by mitosis, and many do so by the asymmetric division process known as budding. Yeasts do not form a single taxonomic or phylogenetic grouping. The term “yeast” is often taken as a synonym for Saccharomyces cerevisiae, but the phylogenetic diversity of yeasts is shown by their placement in two separate phyla: the Ascomycota and the Basidiomycota. The budding yeasts (“true yeasts”) are classified in the order Saccharomycetales. The species are: Arxula adeninivorans (Blastobotrys adeninivorans), Candida boidinii, Schizosaccharomyces pombe, Saccharomyces cerevisiae, Saccharomyces carlsbergensis, Saccharomyces uvarum, Candida utilis, Candida albicans, Saccharomyces boulardii, Brettanomyces bruxellensis, Hansenula polymorpha (Pichia angusta), Pichia pastoris, Kluyveromyces lactis, Yarrowia lipolytica, and Malassezia furfur.

Media for Pathogenic Single-Celled Eukaryotes (May Also be Referred to as Protozoans):

Entamoeba histolytica; Plasmodium; Giardia lamblia; and Trypanosoma brucei.

### Definitions Relating to the Methods, in which the Culture Plate System of the Invention can be Used

The term “discrete colony” as used herein means that a colony is present on a solid medium, which stems from a single colony-forming unit (CFU, that has formed the respective colony), and which is sufficiently far away from at least one further colony (i.e. there is a sufficient distance to said further colony) such that there is no (partial) overgrowth of these at least two colonies. Such a discrete colony has usually the shape of a hemisphere.

The term “automated” as used herein refers to a situation where it is not necessary to carry out steps of the process, in particular the plating step, by hands, i.e. manually. To this aim, in particular a suitable device(s) (such as (a) robot(s) and/or plating devices is used. It can also be automated (e.g. by using a robot) to provide said culture plate system in a suitable distance from the plating device, to then remove said solid medium after plating/dispensing, and to transfer said system to a destination area for carrying the incubation.

The term “exogenous” in combination with nucleic acid as used herein relates to nucleic acid that differs from nucleic acid naturally found and present (“endogenous”) in the microorganisms or cells as used in the present method. In other words, this “exogenous” nucleic acid originates outside the respective microorganisms or cells.

The term “DNA” as used herein is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers or analogs thereof which are by themselves composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single-stranded or double-stranded. In the double stranded form, the nucleotides of the first strand typically hybridize with the nucleotides of the second strand, e.g. by A/T-base-pairing and G/C-base-pairing.

The term “RNA” as used herein is the usual abbreviation for ribonucleic acid. It is a nucleic acid molecule, i.e. a polymer consisting of nucleotide monomers. These nucleotides are usually adenosine-monophosphate, uridine-monophosphate, guanosine-monophosphate and cytidine-monophosphate monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA-sequence. Usually RNA may be obtainable by transcription of a DNA-sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) sequence. In addition to messenger RNA, several non-coding types of RNA exist which may be involved in regulation of transcription and/or translation, and immunostimulation.

The term “insert” as used herein refers to a specific exogenous nucleic acid sequence comprised in the exogenous nucleic acid. This sequence varies depending on the aim of the experiment, but the concept of selectivity based on the presence of an insert is in almost all cases that a specific functionality (e.g. the production of a specific functional part of an enzyme or the whole functional enzyme) is coupled to the insertion of the insert at the intended place at the exogenous nucleic acid.

The term “transformation”/“transforming” as used herein relates to a process, where exogenous nucleic acid is introduced into a microorganism. Typically, the receiving microorganisms are made competent for receiving the exogenous nucleic acid in order to increase the transfer rate. Usually, the process of making microorganisms artificially competent for receiving exogenous nucleic acid involves making the cell passively permeable to nucleic acid by exposing it to conditions that do not normally occur in nature. One way of achieving this is the incubation in a solution containing divalent cations (often calcium chloride) under cold conditions, before being exposed to a heat pulse (heat shock). Alternatively, electroporation is used. The way of making microorganism competent also depends on the type of microorganisms used. Thus, if e.g. E. coli strains are used, both of the afore-mentioned methods can be applied. For yeasts, such as e.g. S. cerevisiae, electroporation is typically used. Alternatively, yeast transformation may be based on the use of lithium acetate, polyethylene glycol, and single-stranded nucleic acid. Typical transformation protocols are known to the skilled person.

The term “transduction”/“transducing” as used herein is also a process, where exogenous nucleic acid is introduced into a microorganism or a eukaryotic cell. Transduction is the process by which nucleic acid is transferred into a microorganism or eukaryotic cell by a virus or via a viral vector. Usually, this involves the use of bacteriophages and is therefore sometimes also referred to as “infection”/“infecting”. Typical transduction protocols are known to the skilled person.

The term “transfection” as used herein relates to a process, where exogenous nucleic acid is introduced into a eukaryotic cell. Transfection of eukaryotic cells typically involves opening transient pores or “holes” in the cell membrane to allow the uptake of exogenous nucleic acid. Transfection can be carried out using calcium phosphate, by electroporation, by cell squeezing or by mixing a cationic lipid with the material to produce liposomes, which fuse with the cell membrane and deposit their cargo inside. Typical transfection protocols are known to the skilled person.

The term “DNA plasmid” as used herein refers to a circular nucleic acid molecule, preferably to an artificial nucleic acid molecule. Such plasmid DNA constructs may be storage vectors, expression vectors, cloning vectors, transfer vectors etc. Preferably, a plasmid DNA within the meaning of the present invention comprises in addition to the elements described herein a multiple cloning site, optionally a selection marker, such as an antibiotic resistance factor, and a sequence suitable for multiplication of the vector, such as an origin of replication. Typical plasmid backbones are e.g. pUC18, pUC19 and pBR322.

The term “bacteriophage” is used herein in the meaning as commonly understood by the skilled person. Thus, reference is made to an organism that infects and replicates within a bacterium. A bacteriophage is composed of proteins that encapsulate a DNA or RNA genome, wherein the genome typically encodes as few as four genes, and as many as hundreds genes.

The term “cosmid” is used herein in the meaning as commonly understood by the skilled person. A “cosmid” is usually defined as a hybrid plasmid that contains a Lambda phage cos sequence (cos sites+plasmid=cosmid). Cosmids are often used as a cloning vector in genetic engineering. Cosmids can be used to build genomic libraries since they can contain rather large DNA sequences, such as 37 to 52 kb of DNA. Cosmids usually replicate as plasmids since they have a suitable origin of replication and frequently also contain a gene for selection. Unlike plasmids, cosmids can also be packaged in phage capsids, which allows the foreign genes to be transferred into or between cells by transduction.

The term “artificial chromosome” as used herein is a DNA construct, which contains genes that promote the even distribution of plasmids after cell division. Usually, reference is made to “BACs”, “bacterial artificial chromosomes, and “YACs”, “yeast artificial chromosomes”. BACs usually have an insert with a size of 150 to 350 kbp and may e.g. be used for sequencing the genome of organisms in genome projects. A short piece of the organism's DNA is amplified as an insert in BACs, and then sequenced. YACs are genetically engineered chromosomes derived from the DNA of the yeast, which is then ligated into a bacterial plasmid. By inserting large fragments of DNA, from 100 to 1000 kb, the inserted sequences can be cloned and physically mapped using a process called chromosome walking. YACs usually contain an autonomously replicating sequence (ARS), centromere and telomeres and a selection marker.

A “determination of the presence of microorganisms or cells comprised in a solution” is particularly relevant for diagnostic purposes, namely to find out whether specific microorganisms or cells are present in a solution. Such a solution is preferably a sample as indicated above, if the presence and/or quantity of microorganisms should be determined. If the sample is e.g. food, it may be determined whether specific microorganisms (such as e.g. pathogens) are present in said sample. This also applies to a situation where the solution is e.g. blood, and the purpose is to determine whether microorganisms of a specific pathogen are present in the blood. As regards the determination of the presence and/or quantity of cells, this mainly relates to specific stem cell populations and cancer stem cells (CSCs).

A “determination of the quantity of microorganisms or cells comprised in a solution” is particularly relevant if it known from other analyses that specific microorganisms or cells are present in a solution, but it is not known, in which quantity said microorganisms or cells are present. Thus, e.g. for specific microorganisms in food or for specific CSCs of a cancer type, there may be a threshold concentration for said microorganisms or cells. This may have an impact on the consumption of the food or the further cancer therapy.

### Detailed Description of the Findings Underlying the Present Invention

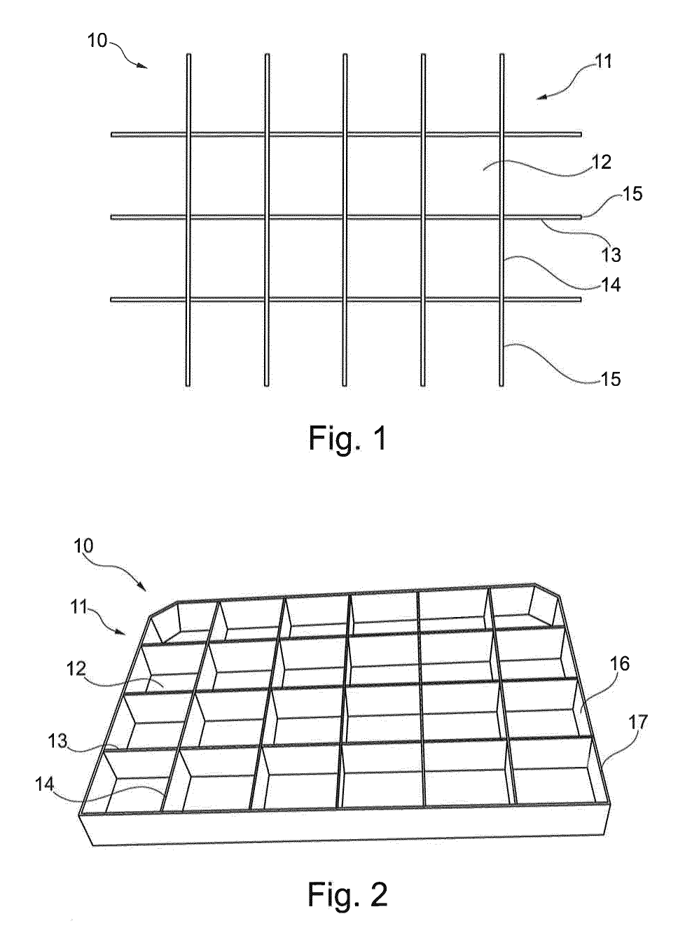

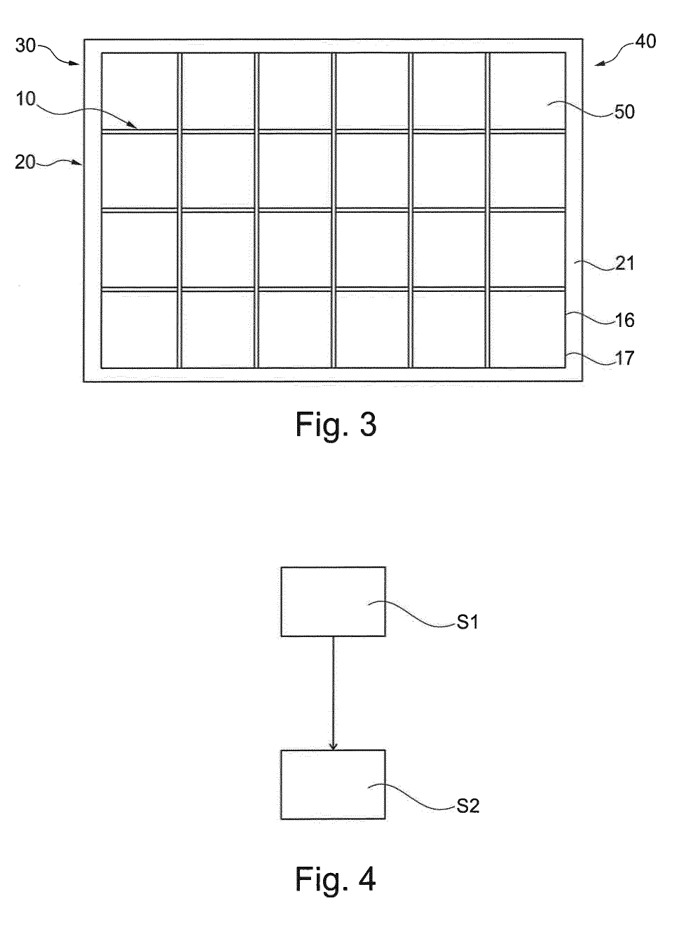

FIG. 1 shows a schematic illustration of an inlay 10 for a culture plate 20 for microorganisms or cells according to the invention. FIG. 2 shows a schematic 3D illustration of another inlay 10 according to the invention. FIG. 3 shows a schematic illustration of a culture plate unit 30 for microorganisms or cells according to the invention.

As shown in FIGS. 1 to 3, the inlay 10 comprises a grid structure 11. The grid structure 11 forms and comprises a plurality of openings 12. The openings 12 are through holes. The openings 12 are angled and in particular essentially square. The square openings 12 allow that a larger portion of the total surface area of the culture plate 20 is useable compared to conventional circular opening. This leads to a reduced consumption of culture plates 20 and less consumable waste. Further, a likelihood to obtain a sufficient number of discrete colonies in one well is increased and an effective area used for plating and picking of colonies is strongly increased.

As shown in FIGS. 1 to 3, all openings 12 are similar in size and shape. “Similar in size” and “similar in shape” comprise also the embodiment of FIG. 2, in which two openings 12 differ from the bulk of openings 12 by a chamfer. The chamfer is explained further below.

As shown in FIG. 3, the grid structure 11 of the inlay 10 is fitted and matches into the culture plate 20. The grid structure 11 partitions the culture plate 20 into an array of 24 discrete wells. The 24 wells or openings 12 of the inlay 10 are regularly distributed relative to the culture plate 20. The wording “regularly distributed” means that the openings 12 form a regular pattern in and on the culture plate 20. This is here achieved by providing the openings 12 with similar distances between centre points of adjacent openings 12.

As shown in FIGS. 1 to 3, the grid structure 11 comprises longitudinal struts 13 and transversal struts 14. The longitudinal struts 13 and transversal struts 14 partition the culture plate 20 into an array of discrete wells. In FIG. 1, face sides 15 of the longitudinal struts 13 and transversal struts 14 are configured to contact sidewalls 21 of the culture plate 20 when the grid structure 11 is fitted into the culture plate 20. In FIG. 2, the grid structure 11 comprises longitudinal struts 13 and transversal struts 14 and additionally a frame unit 16 to partition the culture plate 20 into an array of discrete wells. The frame unit 16 surrounds the grid formed by the longitudinal struts 13 and transversal struts 14. Lateral walls 17 of the frame unit 16 are configured to contact sidewalls 21 of the culture plate 20 when the grid structure 11 is fitted into the culture plate 20. In both cases, the grid structure 11 is unitarily made as one piece. This means, the grid structure 11 may be taken as such or as one piece to be inserted in or removed from the culture plate 20. As a result, the inlay 10 is removeable from the culture plate 20 and may therefore be re-used in the same or another culture plate 20.

The inlays 10 shown in FIGS. 1 to 3 comprise twenty-four openings 12, wherein each (FIG. 1 and FIG. 3) or most (FIG. 2) of the openings 12 have a length of about 18.5 mm and a width of about 17.6 mm. The wall thickness of the grid structure 11 may be about 0.7 mm. The grid structure 11 may be made of polyamide. The following table shows further exemplary dimensions (width and length) of openings in inlays with 6, 12, 24, 48 or 96 openings or wells for different wall thicknesses between 0.5 and 1 mm and e.g. a wall height of 10 mm. Of course, an increasing thickness of an inlay's wall between two openings leads to a decreasing usable surface of the inlay's opening.

| | | | wall thickness | | wall thickness | | wall thickness | |
|---|---|---|---|---|---|---|---|---|
| wells/ | | | 0.5 mm | | 0.7 mm | | 1.0 mm | |
| openings | rows | columns | width | length | width | length | width | length |
| 6 | 2 | 3 | 36.25 | 38.00 | 35.95 | 37.73 | 35.50 | 37.33 |
| 12 | 3 | 4 | 24.00 | 28.38 | 23.73 | 28.13 | 23.33 | 27.75 |
| 24 | 4 | 6 | 17.88 | 18.75 | 17.63 | 18.52 | 17.25 | 18.17 |
| 48 | 6 | 8 | 11.75 | 13.94 | 11.52 | 13.71 | 11.17 | 13.38 |
| 96 | 9 | 12 | 7.67 | 9.13 | 7.44 | 8.91 | 7.11 | 8.58 |

As shown in FIG. 2, the grid structure 11 comprises two chamfers as orientation elements to contact the culture plate 20. These orientation elements allow an unequivocal orientation of the inlay 10 relative to the culture plate 20.

FIG. 3 shows a culture plate unit 30 comprising the culture plate 20 and the inlay 10. In case a medium 50 for growing a culture of microorganisms or cells is filled into the culture plate 20, FIG. 3 also shows a culture plate system 40 for microorganisms or cells comprising the culture plate 20, the inlay 10 and the medium 50. The culture plate system 40 may further comprise a culture.

FIG. 4 shows a schematic illustration of a method for preparing a culture plate system 40 for microorganisms or cells according to the invention. The method comprises the following steps:
In step S1, filling a culture plate 20 with a fluid medium 50.
In step S2, inserting an inlay 10 into the culture plate 20.

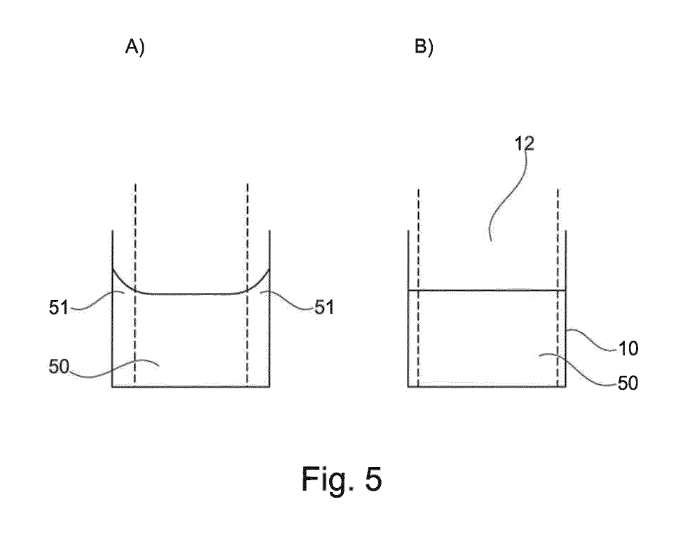

The inlay 10 is inserted into the culture plate 20 filled with medium 50 at a temperature near solidification of the medium 50 to overcome a drawback of commercially available culture plates with circular wells, which is the fact that bulges 51 of medium 50 (e.g., agar medium) are formed at the transition between the medium 50 and the wall of the opening or well. As shown in FIG. 5A, in the prior art, such bulges 51 are caused by shrinking of the hot medium 50 filled into the wells or openings and subsequent drying of the medium 50 during the solidification process. The bulges 51 may cause unfavourable artefacts in the images used by e.g. image assisted colony detection and/or picking devices often used in HT-cloning procedures. As shown in FIG. 5B, this drawback is overcome according to the invention by placing the inlay 10 into the culture plate 20 at a lower temperature just before solidification leading to less subsequent shrinking once the medium 50 sticks to the walls. The temperature just before or near solidification of the medium 50 may be between 30 and 55° C. or between 40 and 45° C. As a result, optical artefacts generated by e.g. agar bulges are minimized which optimizes a visual detection of e.g. colonies using image assisted picking devices.

In view of the medium 50, agarose may be used as a polymerization compound in e.g. bacteriology, because agarose is usually not degraded by bacteria. Agar is a solid gel at room temperature, remaining firm at temperature as high as 65° C. Agar melts at approximately 85° C., a different temperature from that at which it solidifies at about 40 to 45° C. Agar is generally resistant to shear forces; however, different agars may have different gel strengths or degrees of stiffness. Agar is typically used in a final concentration of 1-2% for solidifying culture media. Smaller quantities (0.05-0.5%) are used in media for motility studies (0.5% w/v) and for growth of anaerobes (0.1%) and microaerophiles.

Alternatively, gelatin can also be used as a polymerizing compound. There are several bacteriological assays relating to the ability of certain bacterial strains to “eat” gelatin (gelitinase). Gelatin solidifies when cold 15° C./60° F. and melts at 25° C.-40° C./77° F.-104° F. Historically, gelatin plates were the first solid-medium plates invented by Robert Koch 1881. Some other alternatives comprise starch, carrageen, guar gum, alginate, ficoll, gum katira, isubgol, phytagel. Some of the listed components are used in the context of plant-tissue cultures. It is also possible to use Methylcellulose-based semi-solid media. Such a medium is often used for cell cultures.

### EXAMPLES

The following Examples are merely illustrative and shall describe the present invention in a further way. These Examples shall not be construed to limit the present invention thereto.

### Example 1: Preparation of 24-Well Plates Filled with Solid LB-Agar Using the Inventive Inlay 10

In the present example, the inventive inlay was used to generate wells filled with solid agar, wherein the agar in each well had exactly the same height, and wherein the formation of agar bulges at the well margins was strongly reduced. These characteristics (same agar height; reduced agar bulges) are particularly relevant in the context of HT-cloning, screening and colony picking, in particular if a picking robot is used.

### Preparation of Liquid LB Medium:

12.5 g LB-medium powder (AppliChem) was dissolved in 500 ml ELGA water and autoclaved for 4 hours at 120° C. After the autoclaved LB-medium was tempered (at around 50° C.), antibiotics were added 500 μl ampicillin (100 mg/ml stock solution). In addition to that, a substrate that allows for blue/white selection (such as in particular X Gal) of positive bacterial clones was added (1000 μl of a 20 mg/ml stock; Thermo Scientific) and stirred for 3 minutes at 1100 rpm. The liquid agar was kept at a constant temperature of 50° C. in a water bath.

### Preparation of 24 Well Plates Using the Inventive Inlay:

Under a laminar airflow bench, 35 ml of the prepared liquid agar were carefully dispensed in the middle of a commercially available Omni Tray plate (PS, sterile, Nunc™), avoiding the formation of air-bubbles. After 20 seconds, while the medium was still liquid, the inventive (pre-warmed) inlay was set into the tray. The agar medium was allowed to solidify and plates were stored at 4° C. before bacterial cultures were plated.

### Result:

Using the inventive inlay, 24 well plates with rectangular wells, evenly filled (in the meaning of all wells having the same agar height and all wells having a planar surface including the areas at the well margins) with solid LB agar can be produced. This is of particular importance in the context of HT cloning procedures (e.g., plating, screening, colony picking) since these procedures can be carried out in a more economic fashion and in parallel allow for a higher number of discrete colonies: the inlays can be re-used, the surface area of the wells is larger than in conventional wells and the surface is planar such that also the areas at the well margins can be used for plating, screening and in particular colony picking.

### Example 2: Plating of Bacterial Cultures on 24 Well Plates Prepared Using the Inventive Inlay

The goal of this experiment was to compare the plating efficiency between commercially available 24-well plates with circular wells (Nunc) and culture plates generated with the inventive inlay, namely as described in Example 1.

### Plating of Bacterial Cultures:

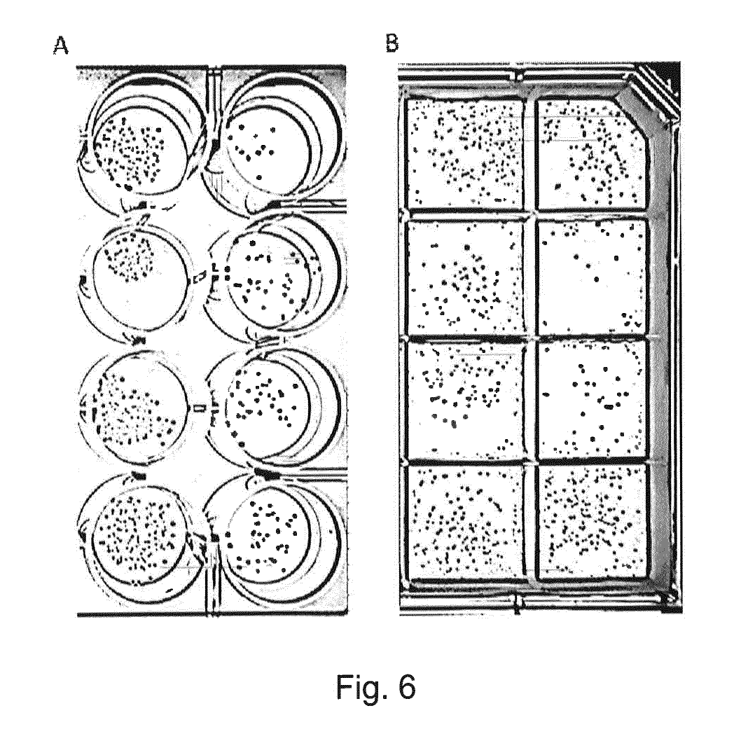

A bacterial culture (Escherichia coli), previously transformed with a plasmid conferring an ampicillin resistance, was cultured in liquid LB ampicillin medium and grown at 37° C. to an optical density of 1.36. Certain dilutions were prepared and used for the plating on two types of 24 well plates: cultures were plated on 24 well culture plates prepared using the inventive inlay (according to Example 1) and on commercially available 24-well plates (each round well containing 800 μl LB Agar with ampicillin). For the round 24 well plates, 30 μl of the respective dilutions were plated (30 μl on a 189 mm² well surface); for the rectangular wells, 50 μl of the respective dilutions were plated (50 μl on a 313 mm² well surface). After plating of the bacterial cultures, the culture plates were incubated for 16 hours at 37° C. Respective images of the culture plates after incubation are shown in FIG. 6. FIG. 6A shows two columns of a commercially available 24 well plate comprising round wells. FIG. 6B shows two columns of a rectangular 24 well culture plate prepared using the inventive inlay (according to Example 1). The same dilutions of bacterial cultures were plated for both plate types. More colonies (black dots) were obtained in FIG. 6B where the inventive inlay was used. Images were taken with the same settings in the same angle (optical artefacts visible in FIG. 6A are not present in FIG. 6B).

### Result:

The results show that an increased volume of bacterial culture per well could be plated on 24 well culture plates (rectangular wells) prepared according to Example 1, without resulting in an overgrowth of bacterial colonies (that is, no single discrete colonies can be detected any more). This is a consequence of the larger surface area that can be exploited for plating of the cultures compared to state-of-the-art 24-well plates (circular wells). The results illustrate the improved surface exploitation that is especially important in the context of HT-cloning procedures. Moreover, it has to be noted that less optical artifacts are produced in the inventive rectangular well culture plates, as can be recognized in FIG. 6B. That, in addition, will improve and simplify the image-based detection of discrete single bacterial colonies e.g., when using a colony picking robot.

1. An inlay for a culture plate for microorganisms or cells comprising a grid structure,
wherein the grid structure is configured to fit into the culture plate,
wherein the grid structure is provided with a plurality of openings, and
wherein the openings are angled.

2. The inlay according to claim 1, wherein the openings are rectangular.

3. (canceled)

4. The inlay according to claim 1, wherein the openings are through holes.

5-8. (canceled)

9. The inlay according to claim 1, wherein the grid structure comprises longitudinal struts and transversal struts, and wherein face sides of the longitudinal struts and transversal struts are configured to contact sidewalls of the culture plate when the grid structure is fitted into the culture plate.

10. (canceled)

11. The inlay according to claim 1, wherein the grid structure comprises a frame unit and wherein lateral walls of the frame unit are configured to contact sidewalls of the culture plate when the grid structure is fitted into the culture plate.

12. (canceled)

13. The inlay according to claim 1, wherein the grid structure is configured to partition the culture plate into an array for discrete wells.

14-23. (canceled)

24. The inlay according to claim 1, wherein the culture plate comprises 96 openings, wherein most of the openings have a length of about 8.6 mm and a width of about 8.1 mm.

25. (canceled)

26. The inlay according to claim 1, wherein the grid structure is made of PMMA, polyamide, polystyrol, acrylic or polycarbonate.

27. The inlay according to claim 1, wherein the grid structure is autoclavable.

28. (canceled)

29. A culture plate unit for microorganisms or cells comprising a culture plate and an inlay inserted into the culture plate, wherein:
the inlay comprises a grid structure;
the grid structure is configured to fit into the culture plate;
the grid structure is provided with a plurality of openings; and
the openings are angled.

30. The culture plate unit according to claim 29, wherein the inlay is removeable from the culture plate.

31. The culture plate unit according to claim 29 further comprising a medium for growing microorganisms or cells.

32. The culture plate unit according to claim 31, wherein the medium is thermosetting.

33. A method for preparing a culture plate system for microorganisms or cells, comprising the steps of:
a) filling a culture plate with a medium, and
b) inserting an inlay into the culture plate,
wherein the inlay comprises a grid structure,
wherein the grid structure is configured to fit into the culture plate,
wherein the grid structure is provided with a plurality of openings, and
wherein the openings are angled.

34. The method according to claim 33, wherein the inlay is inserted into the culture plate filled with medium at a temperature near solidification of the medium.

35. The method according to claim 34, wherein the temperature near solidification of the medium is between 40 and 45° C.

36. (canceled)

37. A method for obtaining at least one discrete colony from microorganisms or cells comprised in a solution, wherein the method comprises:
a) filling a culture plate with a medium, and
b) inserting an inlay into the culture plate, wherein:
the inlay comprises a grid structure,
the grid structure is configured to fit into the culture plate,
the grid structure is provided with a plurality of openings,
the openings are angled, and
the at least one discrete colony is obtained from microorganisms during high throughput cloning.

38. (canceled)

39. The method according to claim 37, wherein the at least one discrete colony is obtained from cells during transfection of eukaryotic cells.

40. The method according to claim 37, further comprising determining the presence and/or quantity of microorganisms or cells potentially comprised in a solution.

41. The method according to claim 37, wherein the method is an automated method.
